(19) United States
(12) Patent Application Publication
(10) Pub. No.: US 2024/0041080 A1
(43) Pub. Date: Feb. 8, 2024

(54) METHOD FOR PURIFYING A CRUDE COMPOSITION COMPRISING ONE OR MORE STEVIOL GLYCOSIDES

(71) Applicant: CORN PRODUCTS DEVELOPMENT, INC., WESTCHESTER, IL (US)

(72) Inventors: Jennifer BRIDGE, Bridgewater, NJ (US); Kaitlin DIBLASIO, Bridgewater, NJ (US); Sarfaraz PATEL, Bridgewater, NJ (US)

(21) Appl. No.: 18/266,718

(22) PCT Filed: Dec. 15, 2021
(86) PCT No.: PCT/US2021/063615

§ 371 Date: Jun. 12, 2023

Related U.S. Application Data
(60) Provisional application No. 63/126,366, filed on Dec. 16, 2020.

(51) Int. Cl. A23L 27/30; A23L 2/60; C07H 1/06; C07H 15/256

(52) U.S. Cl. CPC A23L 27/36; A23L 2/60; C07H 1/06; C07H 15/256

(57) ABSTRACT

Disclosed herein is a method for purifying a crude composition comprising one or more steviol glycosides, compositions obtained therefrom and compositions in amorphous form comprising one or more steviol glycosides.

Disclosed herein is a method for purifying a crude composition comprising one or more steviol glycosides, compositions obtained therefrom and compositions in amorphous form comprising one or more steviol glycosides.

Steviol glycosides are sweet non-caloric compounds which are commonly used to sweeten foods and beverages in which want to limit the caloric value. They can be isolated from the leaves of the Stevia rebaudiana plant, commonly referred to as the Stevia plant.

Known steviol glycosides that can be isolated from Stevia leaves include Stevioside, Rebaudioside A, Rebaudioside B, Rebaudioside C, Rebaudioside D, Rebaudioside E, Rebaudioside F, Rebaudioside I, Rebaudioside J, Rebaudioside M (also referred to as Rebaudioside X), and Steviolbioside, of which Stevioside and Rebaudioside A are present at the highest concentrations.

Rebaudioside B, Rebaudioside D and Rebaudioside M are present in substantially smaller amounts. However, their taste profile is generally preferred, since they produce less bitterness and/or after taste than Stevioside and Rebaudioside A.

As different steviol glycosides are present in the leaves of the Stevia plant, the recovery process generally involves recovering a mixture comprising different steviol glycosides. Such mixture can be purified by various known combinations of unit processes to increase the concentration of the steviol glycoside(s) of interest and to remove compounds other than steviol glycosides. Currently, the health authorities often require a total steviol glycoside concentration of at least 95 wt. % on a dry weight basis.

WO 2011/112892 describes a process for obtaining high purity Rebaudioside D. The method involves passing a solution of steviol glycosides through a multi-column system including columns with a packed resin. It is described that impurities are removed by eluting the multi-column system with an aqueous alcohol solution. The method may further involve eluting fraction high in Rebaudioside D with an aqueous alcohol solution, and purifying the Rebaudioside D fractions by multiple crystallizations.

The method of WO 2011/112892 involves the removal of impurities (components other than steviol glycosides) using an elution step in a column using a washing solution. Thus, a separate step is applied for removing such impurities. Crystallization is typically a time consuming process and requires specialized equipment to maintain required temperature and other experimental conditions to obtain desired product purity. Furthermore, crystalline product is relatively difficult to dissolve.

WO 2014/098833 describes a method for preparing amorphous Rebaudioside X (also referred to as Rebaudioside M). Rebaudioside X is purified by multiple crystallizations first, after which it is subjected to the process to convert the Rebaudioside X into the amorphous form. It is further described that Rebaudioside X in amorphous form has improved dissolution characteristics compared to the crystalline form. As the method of WO 2014/098833 requires multiple crystallization steps prior to obtaining the amorphous product, the method is relatively complex.

In view of the above, there is a need for an efficient purification process that enables an increase in the total steviol glycoside content of a composition in amorphous form.

Furthermore, there is a need for an efficient purification process enables an increase in the content of Rebaudioside B, Rebaudioside D, and/or Rebaudioside M of a composition in amorphous form.

The present invention relates to a method for purifying a crude composition comprising one or more steviol glycosides, said method comprising:

providing a crude composition comprising one or more steviol glycosides, wherein said composition is in amorphous form;
subjecting said crude composition to a trituration step, said trituration step comprising contacting said crude composition with a polar solvent, resulting in a triturated product; and recovering said triturated product.

The invention further relates to a method for purifying a crude composition comprising one or more steviol glycosides, said method comprising:

providing a crude composition comprising one or more steviol glycosides by spray drying a solution comprising said one or more steviol glycosides;
subjecting said crude composition to a trituration step, said trituration step comprising contacting said crude composition with a polar solvent, resulting in a triturated product; and recovering said triturated product.

The invention further relates to a composition in amorphous form obtainable by the method according to the invention.

The invention further relates to a composition in amorphous form comprising:
from 10 to 60 wt. % of Rebaudioside D on a dry weight basis;
from 5 to 30 wt. % of Rebaudioside M on a dry weight basis; and
from 1 to 60 wt. % of Rebaudioside A on a dry weight basis.

The invention further relates to the use of the composition according to the invention as an ingredient for a food product, for example a beverage.

The invention further relates to a method for preparing a food product, for instance a beverage, said method comprising incorporating the composition according to the invention in said food product.

### BRIEF DESCRIPTION OF THE FIGURES

FIG. 1 is a schematic drawing of one embodiment of the method according to the invention.

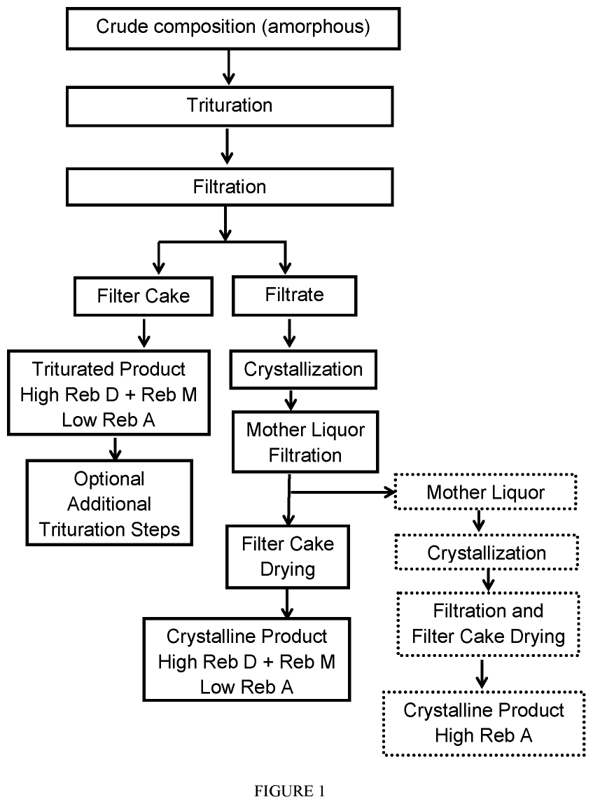

Providing a Crude Composition Comprising One or More Steviol Glycosides

The method according to the invention comprises providing a crude composition comprising one or more steviol glycosides. According to an aspect of the invention, the crude composition is the amorphous form. According to a further aspect of the invention, the crude composition is obtained by spray drying a solution comprising the one or more steviol glycosides in dissolved form.

The skilled person is well aware how to distinguish an amorphous form and a crystalline form. For instance, X-ray diffraction (XRPD) and scanning electron microscopy (SEM) can suitably be used to establish the presence of the amorphous form.

The crude composition in the amorphous form may be obtained in any suitable manner. Supercooling of liquids, milling, lyophilization spray drying, dehydration of crystalline hydrates are known methods may suitably be used. It is also possible to use the method described in WO 2014/098833. In one embodiment, the crude composition in amorphous form is obtained by spray drying a solution comprising the one or more steviol glycosides in dissolved form.

The crude composition comprises one or more steviol glycosides. The crude composition may, for instance, comprise Stevioside, Rebaudioside A, Rebaudioside B, Rebaudioside C, Rebaudioside D, Rebaudioside F, Rebaudioside I, Rebaudioside J, Rebaudioside M, Dulcoside A, Rubusoside or a mixture of one or more of these steviol glycosides.

The crude composition may, for instance, comprise Rebaudioside B. In one embodiment, the crude composition comprises at least 5 wt. % of Rebaudioside B on a dry weight basis. In another embodiment, the crude composition comprises at least 10 wt. % of Rebaudioside B on a dry weight basis. There is no particular upper limit for the content of Rebaudioside B in the crude composition. The crude composition may, for instance, comprise less than 90 wt. % of Rebaudioside B on a dry weight basis or less than 60 wt. % of Rebaudioside B on a dry weight basis.

The crude composition may, for instance, comprise Rebaudioside D. In one embodiment, the crude composition comprises at least 5 wt. % of Rebaudioside D on a dry weight basis. In another embodiment, the crude composition comprises at least 10 wt. % of Rebaudioside D on a dry weight basis. There is no particular upper limit for the content of Rebaudioside D in the crude composition. The crude composition may, for instance, comprise less than 90 wt. % of Rebaudioside D on a dry weight basis or less than 60 wt. % of Rebaudioside D on a dry weight basis.

The crude composition may, for instance, comprise Rebaudioside M. In one embodiment, the crude composition comprises at least 5 wt. % of Rebaudioside M on a dry weight basis. In another embodiment, the crude composition comprises at least 10 wt. % of Rebaudioside M on a dry weight basis. There is no particular upper limit for the content of Rebaudioside M in the crude composition. The crude composition may, for instance, comprise less than 90 wt. % of Rebaudioside M on a dry weight basis or less than 60 wt. % of Rebaudioside M on a dry weight basis.

The crude composition may, for instance, comprise Rebaudioside A. In one embodiment, the crude composition comprises at least 10 wt. % of Rebaudioside A on a dry weight basis. In another embodiment, the crude composition comprises at least 20 wt. % of Rebaudioside A on a dry weight basis. There is no particular upper limit for the content of Rebaudioside M in the crude composition. The crude composition may, for instance, comprise less than 90 wt. % of Rebaudioside A on a dry weight basis or less than 60 wt. % of Rebaudioside A on a dry weight basis.

Preferably, the crude composition has a total content of steviol glycosides of at least 60 wt. % on a dry weight basis. In one embodiment, the crude composition may for instance have a total content of steviol glycosides of from 60 to 98 wt. % based on a dry weight basis. In another embodiment, the crude composition may for instance have a total content of steviol glycosides of from 60 to 95 wt. % based on a dry weight basis.

In one embodiment, the one or more steviol glycosides are obtained from the Stevia rebaudiana plant. The crude composition comprising the one or more steviol glycosides and/or the solution comprising the one or more steviol glycosides may be obtained from the Stevia rebaudiana plant by methods known to the skilled person. A crude composition comprising one or more steviol glycosides or a solution comprising one or more glycosides may for instance be obtained by a process comprising subjecting leaves of the Stevia rebaudiana plant to extraction with an (aqueous) solvent, followed by flocculation, filtration, adsorption and elution, ion exchange or a combination thereof. A solution resulting from such process may be subjected to spray drying. An exemplary processes is described in A. Németh, Extraction of steviol glycosides from dried Stevia rebaudiana by pressurized hot water extraction, Acta Alimentaria , Vol. 48(2) (2019) p. 241-252.

Subjecting the Crude Composition to a Trituration Step, said Trituration Step Comprising Contacting the Crude Composition with a Polar Solvent, Resulting in a Triturated Product

The method according to the invention comprises subjecting the crude composition to a trituration step. The trituration step comprises contacting the crude composition with a polar solvent.

The trituration unit operation as such is well-known to the skilled person and is used in organic chemistry to purify crude chemical compounds containing soluble impurities. A solvent may be chosen in which the desired product has a relatively low solubility and the undesired by-products have a relatively high solubility.

Thus, trituration differs from purification by crystallization where the desired product is dissolved first and subsequently recrystallized. Purification by crystallization results in a crystalline product. Subjecting an crude composition in amorphous form results in a triturated product in amorphous form.

In one embodiment, contacting a crude amorphous composition comprising one or more steviol glycosides with a polar solvent enables a triturated product having an increased total content of steviol glycosides to be obtained.

In another embodiment, the total steviol glycoside content in the triturated product on a dry weight basis is at least 3 wt. % (absolute) higher than the total steviol glycoside content in the crude composition on a dry weight basis. In another embodiment, the total steviol glycoside content in the triturated product on a dry weight basis is at least 4 wt. % (absolute) higher than the total steviol glycoside content in the crude composition on a dry weight basis. In still another embodiment, the total steviol glycoside content in the triturated product on a dry weight basis is at least 5 wt. % (absolute) higher than the total steviol glycoside content in the crude composition on a dry weight basis.

The skilled person will understand that, as used herein, a difference expressed in percentage (absolute) refers to the difference in percent point. By way of illustration, in an exemplary embodiment where the crude composition comprises 10 wt. % of Rebaudioside M and the triturated product comprises 15 wt. % of Rebaudioside M, the content of Rebaudioside M in the triturated product is 5 wt. % (absolute) higher than the content of Rebaudioside M in the crude composition.

In some embodiments, the triturated product has a total steviol glycoside content of greater than 95 wt. % based on a dry weight basis. In still other embodiments, the triturated product has a total steviol glycoside content of from 95 wt. % to 99 wt. % based on a dry weight basis.

In still even further embodiments, contacting a crude amorphous composition comprising a steviol glycoside selected from Rebaudioside B, Rebaudioside D and Rebaudioside M with a polar solvent provides a triturated product having an increased content of these steviol glycosides.

In one embodiment, the content of Rebaudioside B in the triturated product on a dry weight basis is at least 5 wt. % (absolute) higher than the content of Rebaudioside B in the crude composition on a dry weight basis. In another embodiment, the content of Rebaudioside B in the triturated product on a dry weight basis is at least 10 wt. % (absolute) higher than the content of Rebaudioside B in the crude composition on a dry weight basis. In still a further embodiment, the content of Rebaudioside B in the triturated product on a dry weight basis is at least 15 wt. % (absolute) higher than the content of Rebaudioside B in the crude composition on a dry weight basis.

In one embodiment, the content of Rebaudioside D in the triturated product on a dry weight basis is at least 5 wt. % (absolute) higher than the content of Rebaudioside D in the crude composition on a dry weight basis. In another embodiment, the content of Rebaudioside D in the triturated product on a dry weight basis is at least 10 wt. % (absolute) higher than the content of Rebaudioside D in the crude composition on a dry weight basis. In another embodiment, the content of Rebaudioside D in the triturated product on a dry weight basis is at least 15 wt. % (absolute) higher than the content of Rebaudioside D in the crude composition on a dry weight basis.

In one embodiment, the content of Rebaudioside M in the triturated product on a dry weight basis is at least 5 wt. % (absolute) higher than the content of Rebaudioside M in the crude composition on a dry weight basis. In still another embodiment, the content of Rebaudioside M in the triturated product on a dry weight basis is at least 10 wt. % (absolute) higher than the content of Rebaudioside M in the crude composition on a dry weight basis. In yet another embodiment, the content of Rebaudioside M in the triturated product on a dry weight basis is at least 15 wt. % (absolute) higher than the content of Rebaudioside M in the crude composition on a dry weight basis.

In one embodiment, a triturated product having a decreased content of Rebaudioside A is obtained by contacting a crude amorphous composition comprising Rebaudioside A with a polar solvent.

In some embodiments, the content of Rebaudioside A in the triturated product on a dry weight basis is at least 5 wt. % (absolute) lower than the content of Rebaudioside A in the crude composition on a dry weight basis. In other embodiments, the content of Rebaudioside A in the triturated product on a dry weight basis is at least 10 wt. % (absolute) lower than the content of Rebaudioside A in the crude composition on a dry weight basis. In still other embodiments, the content of Rebaudioside A in the triturated product on a dry weight basis is at least 15 wt. % (absolute) lower than the content of Rebaudioside A in the crude composition on a dry weight basis.

Any suitable polar solvent may be used. In some embodiments, the solvent may for instance comprise an alcohol, water or a mixture thereof. In further embodiments, the alcohol is C1-C4 alcohol or methanol, ethanol, or a mixture thereof. In still further embodiments, the alcohol is ethanol. Ethanol was found to provide optimal results.

In one embodiment, the solvent comprises at least 5 wt. % of alcohol, such as from 10 wt. % to 90 wt. % of alcohol. In other embodiments, the solvent comprises between 60 and 95 wt. % of alcohol or between 70 and 90 wt. % of alcohol. As used herein the percentages of alcohol refer to the total percentage of alcohols present in the solvent. In some embodiments, the solvent further comprises water. In other embodiments, the sum content of alcohol and water in the solvent is at least 95 wt. %. In still further embodiments the sum content of alcohol and water in the solvent is at least 99 wt. %.

In some embodiments, the solvent comprises at least 5 wt. % of methanol and ethanol, such as from 10 wt. % to 90 wt. % of methanol and ethanol. In yet other embodiments, the solvent comprises between 60 and 95 wt. % of methanol and ethanol. In still further embodiments, the solvent comprises between 70 and 90 wt. % of methanol and ethanol. As used herein the percentages of methanol and ethanol refer to the total percentage of methanol and ethanol present in the solvent. In some embodiments, the solvent further comprises water. In other embodiments, the sum content of methanol and ethanol and water in the solvent is at least 95 wt. %. In yet even further embodiments, the sum content of methanol and ethanol and water in the solvent is at least 99 wt. %.

In further embodiments, the solvent comprises at least 5 wt. % of ethanol, such as from 10 wt. % to 90 wt. % of ethanol. In yet other embodiments, the solvent comprises between 60 and 95 wt. % of ethanol. In yet even further embodiments, the solvent comprises between 70 and 90 wt. % of ethanol. In some embodiments, the solvent further comprises water. In other embodiments, the sum content of ethanol and water in the solvent is at least 95 wt. %. In still other embodiments, the sum content of ethanol and water in the solvent is at least 99 wt. %.

In some embodiments, the trituration step comprises preparing a slurry comprising the crude composition and said solvent, wherein said slurry comprises between 120 and 250 g crude composition per liter of slurry. In other embodiments, said slurry comprises between 140 and 220 g crude composition per liter of slurry. In still other embodiments, said slurry comprises between 160 and 200 g crude composition per liter of slurry. Working within the 140-220 g or 160-200 g ranges was found to enhance the recovery.

In some embodiments, the trituration step is effected at a temperature below 40° C. In other embodiments, the trituration step is effected at a temperature below 35° C. It is possible to perform the trituration at temperatures below room temperature, for instance as low as 2° C., for instance at a temperature between 2° C. and 40° C., for instance at a temperature between 25° C. and 35° C. In one embodiment, the trituration step is effected at a temperature from 20 to 35° C.

Contacting the crude composition with the solvent may be effected for any suitable period. In one embodiment, said contacting is effected for at least 5 minutes. In another embodiment, said contacting is effected for at least 10 minutes. In a further embodiment, said contacting is effected for at least 20 minutes.

In an embodiment of the invention, the trituration step is repeated one or more times. In some embodiments, the triturated product recovered from the trituration step is subjected to one or more further trituration steps.

Recovering the Triturated Product.

Recovering the triturated product may be effected in any suitable manner.

Generally, the trituration step comprises preparing a slurry comprising said crude composition and said solvent. Triturated product may be recovered by separating the triturated product from the slurry, by any suitable solid-liquid separation method, such as, for example, by filtration or centrifugation. In some embodiments, the triturated product is recovered by filtration or centrifugation.

Subjecting Solvent Recovered from the Trituration Step to at Least One Crystallization Step to Obtain Crystalline Rebaudioside B, Crystalline Rebaudioside D, Crystalline Rebaudioside M and/or Crystalline Rebaudioside A (Optional)

In some embodiments, the method according to the invention further comprises (d) recovering the solvent from the trituration step and subjecting said recovered solvent to one or more crystallization steps to obtain crystalline steviol glycosides comprising Rebaudioside B, crystalline Rebaudioside D, crystalline Rebaudioside M, crystalline Rebaudioside A, or mixtures thereof. This is found to increase the overall recovery (yield) of the method. In other embodiments, the method according to the invention further comprises: (e) recovering the mother liquor from the one or more crystallization steps of (d) and subjecting said recovered mother liquor to one or more crystallization steps to obtain crystalline steviol glycosides comprising Rebaudioside A.

In some embodiments, the one or more crystallization steps include a first crystallization step to obtain crystalline Rebaudioside D, crystalline Rebaudioside M, or mixture thereof, and a second crystallization step to obtain crystalline Rebaudioside A from mother liquor recovered from said first crystallization step.

Crystallization of Rebaudioside D, crystalline Rebaudioside M and/or crystalline Rebaudioside A may be effected by methods known to the skilled person.

Subject matter contemplated by the present disclosure is set out in the following numbered embodiments:
1. A method for purifying a crude composition comprising one or more steviol glycosides, said method comprising: providing a crude composition comprising one or more steviol glycosides, wherein said composition is in amorphous form; subjecting said crude composition to a trituration step, said trituration step comprising contacting said crude composition with a polar solvent, resulting in a triturated product; and recovering said triturated product.
2. The method according to embodiment 1, wherein said providing said crude composition comprises spray drying a solution comprising said one or more steviol glycosides in dissolved form.
3. A method for purifying a crude composition comprising one or more steviol glycosides, said method comprising: providing a crude composition comprising one or more steviol glycosides by spray drying a solution comprising said one or more steviol glycosides; subjecting said crude composition to a trituration step, said trituration step comprising contacting said crude composition with a polar solvent, resulting in a triturated product; and recovering said triturated product.
4. The method according to any preceding embodiment, wherein said one or more steviol glycosides comprises Rebaudioside B, Rebaudioside D, Rebaudioside M, or a mixture thereof.
5. The method according to any preceding embodiment, wherein said one or more steviol glycosides comprises Rebaudioside B, wherein said crude composition comprises at least 5 wt. % or at least 10 wt. % of Rebaudioside B on a dry weight basis.
6. The method according to any preceding embodiment, wherein said one or more steviol glycosides comprises Rebaudioside D, wherein said crude composition comprises at least 5 wt. % or at least 10 wt. % of Rebaudioside D on a dry weight basis.
7. The method according to any preceding embodiment, wherein said one or more steviol glycosides comprises Rebaudioside M, wherein said crude composition comprises at least 5 wt. % or at least 10 wt. % of Rebaudioside M on a dry weight basis.
8. The method according to any preceding embodiment, wherein said one or more steviol glycosides comprises Rebaudioside A, wherein said crude composition comprises at least 10 wt. % or at least 20 wt. % of Rebaudioside A on a dry weight basis.
9. The method according to any preceding embodiment, wherein said crude composition has a total content of steviol glycosides of at least 60 wt. % on dry weight basis.
10. The method according to any preceding embodiment, wherein said crude composition has a total content of steviol glycosides of from 60 to 98 wt. % or from 60 to 95 wt. % based on a dry weight basis.
11. The method according to any preceding embodiment, wherein said solvent comprises water, an alcohol, or a mixture thereof.
12. The method according to embodiment 11, wherein the sum content of alcohol and water in the solvent is at least 95 wt. % or at least 99 wt. %.
13. The method according to embodiment 11 or 12, wherein said alcohol is methanol, ethanol, or a mixture thereof.
14. The method according to any one of embodiments 11-13, wherein said alcohol is ethanol.
15. The method according to embodiments 13 or 14, wherein the sum content of alcohol and ethanol in the solvent is at least 95 wt. % or at least 99 wt. %.
16. The method according to any preceding embodiment, wherein said solvent comprises (i) at least 5 wt. % alcohol; (ii) from 10 wt. % to 90 wt. % of alcohol; (iii) between 60 and 95 wt. % of alcohol; or (iv) between 70 and 90 wt. % of alcohol.
17. The method according to any preceding embodiment, wherein said solvent comprises (i) at least 5 wt. % of ethanol; (ii) from 10 wt. % to 90 wt. % of ethanol; (iii) between 60 and 95 wt. % of ethanol; and (iv) between 70 and 90 wt. % of ethanol.
18. The method according to any preceding embodiment, wherein the trituration step comprises preparing a slurry comprising said crude composition and said solvent, wherein said slurry comprises between 120 and 250 g crude composition per liter of slurry, between 140 and 220 g crude composition per liter of slurry, or between 160 and 200 g crude composition per liter of slurry.
19. The method according to any preceding embodiment, wherein the trituration step is effected at a temperature below 40° C. or from 20 to 35° C.
20. The method according to any preceding embodiment, wherein said contacting is effected for at least 5 minutes, at least 10 minutes, or at least 20 minutes.
21. The method according to any preceding embodiment, wherein said triturated product has a total steviol glycoside content of greater than 95 wt. % based on a dry weight basis.
22. The method according to any preceding embodiment, wherein the content of Rebaudioside B in the triturated product on a dry weight basis is at least 5 wt. % (absolute), at least 10 wt. % (absolute), or at least 15 wt. % (absolute) higher than the content of Rebaudioside B in the crude composition on a dry weight basis.
23. The method according to any preceding embodiment, wherein the content of Rebaudioside D in the triturated product on a dry weight basis is at least 5 wt. % (absolute), at least 10 wt. % (absolute), or at least 15 wt. % (absolute) higher than the content of Rebaudioside D in the crude composition on a dry weight basis.
24. The method according to any preceding embodiment, wherein the content of Rebaudioside M in the triturated product on a dry weight basis is at least 5 wt. % (absolute), at least 10 wt. % (absolute), or at least 15 wt. % (absolute) higher than the content of Rebaudioside M in the crude composition on a dry weight basis.
25. The method according to any preceding embodiment, wherein the content of Rebaudioside A in the triturated product on a dry weight basis is at least 5 wt. % (absolute) lower than the content of Rebaudioside A in the crude composition on a dry weight basis, preferably at least 10 wt. % (absolute) lower, more preferably at least 15 wt. % (absolute) lower.
26. The method according to any preceding embodiment, wherein the total steviol glycoside content in the triturated product on a dry weight basis is at least 3 wt. % (absolute), at least 4 wt. % (absolute), or at least 5 wt. % (absolute) higher than the total steviol glycoside content in the crude composition on a dry weight basis.
27. The method according to any preceding embodiment, wherein the trituration step comprises obtaining a slurry comprising said crude composition and said solvent, and wherein said recovering of said triturated product comprises separating said triturated product from said slurry, optionally wherein said triturated product is separated from said slurry by filtration or centrifugation.
28. The method according to any preceding embodiment, wherein the method further comprises: (d) recovering the solvent from the trituration step and subjecting said recovered solvent to one or more crystallization steps to obtain crystalline steviol glycosides comprising crystalline Rebaudioside B, crystalline Rebaudioside D, crystalline Rebaudioside M, crystalline Rebaudioside A, or mixtures thereof.
29. The method according to any preceding embodiment, wherein the method further comprises: (e) recovering the mother liquor from the one or more crystallization steps of (d) and subjecting said recovered mother liquor to one or more crystallization steps to obtain crystalline steviol glycosides comprising Rebaudioside A.
30. The method of embodiment 28 or 29, wherein said one or more crystallization steps comprises a first crystallization step to obtain crystalline Rebaudioside D, crystalline Rebaudioside M, or mixture thereof, and a second crystallization step to obtain crystalline Rebaudioside A from mother liquor recovered from said first crystallization step.
31. A steviol glycoside composition in amorphous form obtainable by the method according to any preceding claim.
32. A steviol glycoside composition in amorphous form comprising:
   from 10 to 60 wt. % of Rebaudioside D on a dry weight basis;
   from 5 to 30 wt. % of Rebaudioside M on a dry weight basis; and
   from 1 to 60 wt. % of Rebaudioside A on a dry weight basis.
33. The steviol glycoside composition according to embodiment 32, wherein the total steviol glycoside content in the composition is at least 95 wt. % on a dry weight basis.
34. Use of the steviol glucoside composition according to any one of embodiments 31 to 33 as an ingredient for a food product, for instance wherein said food product is a beverage.
35. Method for preparing a food product, for instance a beverage, said method comprising incorporating the composition according to any one of embodiments 31 to 33 in said food product.

### EXAMPLES

### Analysis Methods and Examples

As used herein, the content of the various Rebaudiosides in the various products on a dry weight basis is determined by drying the product and analysis by HPLC using the 2017 JECFA monograph for steviol glycosides. The moisture content is determined using the Karl Fischer titration method. In accordance with the JECFA monograph, a moisture level of less than 6% is considered dry product.

In all examples, reported percentages of Rebaudiosides and Total Steviol Glycosides are on a dry weight (anhydrous) basis.

% Recovery of all products is calculated by equation (I) shown below and is reported on a dry weight (anhydrous) basis:

% Recovery of Rebaudioside=(Amount of anhydrous Rebaudioside in product÷Amount of anhydrous Rebaudioside in Feed)* 100 (I)

In the examples amorphous steviol glycoside powder was used which had been prepared as follows. Steviol glycoside powdered raw material was prepared by spray drying. Reb A, D, M and other steviol glycosides were mixed in desired proportion in water, resulting in a composition of spray drying feed on anhydrous basis of 16 wt. % Reb M, 21 wt. % Reb D and 38 wt. % Reb A. The total steviol glycoside content of feed material on anhydrous basis was 94 wt. %. The resulting dispersion was heated to 80-85° C. to dissolve the rebaudiosides and the solution was spray dried at 85-90° C. outlet temperature, the inlet air temperature was controlled between 180-190° C. to obtain desired outlet temperature.

Aqueous ethyl alcohol was used as solvent in examples below. The solvent concentration is reported on weight basis with percent number indicating weight percent of alcohol used in preparation of solvent e.g. 70% ethyl alcohol is prepared by mixing 70 parts by weight ethyl alcohol with 30 parts by weight water.

### EXAMPLE 1

### Trituration with 70% Ethyl Alcohol Solvent

120, 170, & 250 g/L of amorphous steviol glycoside powder (crude composition) having a composition as indicated in Table 1 were mixed with 70% ethyl alcohol as solvent to produce a slurry. The slurry was stirred at 700 rpm for 1 hour at room temperature (25° C.), filtered on a Buchner funnel with a Whatman 5 filter and washed with cold anhydrous 200 proof ethanol.

The resulting powder (triturated product) was dried and analyzed by HPLC using the 2017 JECFA monograph for steviol glycosides. The results are set forth in Table 1. In all instances (Examples 1.1, 1.2, and 1.3), the total steviol glycoside (“TSG”) content, as well as the Reb D content increased as a result of the trituration. In Example 1.3 of Table 1, the Reb M content also increased.

TABLE 1

| | Crude | Triturated Product | | | Triturated Product | | |
|---|---|---|---|---|---|---|---|
| | Composition | Composition (wt. %) | | | Recovery (%) | | |
| | Composition | Ex. 1.1 | Ex. 1.2 | Ex. 1.3 | Ex. 1.1 | Ex. 1.2 | Ex. 1.3 |
| | (wt. %) | (120 g/L) | (170 g/L) | (250 g/L) | (120 g/L) | (170 g/L) | (250 g/L) |
| Reb D | 19.65 | 59.41 | 55.21 | 41.22 | 39.98 | 63.21 | 65.76 |
| Reb M | 15.10 | 7.88 | 8.93 | 27.26 | 4.61 | 9.55 | 48.79 |
| Reb A | 36.10 | 18.88 | 21.13 | 18.29 | 4.71 | 9.77 | 10.03 |

| | | | | | |
|---|---|---|---|---|---|
| TSG* | 89.78 | 94.35 | 93.44 | 93.59 | N/A |

*Composition of other steviol glycosides e.g. Stevioside, Rebaudioside F, Rebaudioside C, Dulcoside A, Rubusoside, Rebaudioside B are not reported in Table 1 but are included in TSG.

### EXAMPLE 2

### Trituration with 80% Ethyl Alcohol Solvent

Example 1 was repeated (120 g/L & 170 g/L) with the difference that 80% ethyl alcohol (balance water) was used instead of 70% ethyl alcohol. The results are set forth in Table 2. In all instances, the Reb D, Reb M, and TSG content increased as a result of the trituration. The recovery of Reb D and Reb M was substantially higher compared to Example 1.

TABLE 2

| | Crude | Triturated Product | | Triturated Product | |
|---|---|---|---|---|---|
| | Composition | Composition (wt. %) | | Recovery (%) | |
| | Composition | Ex. 2.1 | Ex. 2.2 | Ex. 2.1 | Ex. 2.2 |
| | (wt. %) | (120 g/L) | (170 g/L) | (120 g/L) | (170 g/L) |
| Reb D | 19.65 | 51 | 46 | 83.48 | 89.06 |
| Reb M | 15.10 | 17 | 27 | 36.95 | 68.57 |
| Reb A | 36.10 | 20 | 17 | 17.51 | 19.40 |

| | | | | |
|---|---|---|---|---|
| TSG* | 89.78 | 97 | 96 | N/A |

*Composition of other steviol glycosides e.g. Stevioside, Rebaudioside F, Rebaudioside C, Dulcoside A, Rubusoside, Rebaudioside B are not reported in Table 2 but are included in TSG.

### EXAMPLE 3

### Trituration with 87.5% Ethyl Alcohol Solvent

Example 1 was repeated with the difference that 87.5% ethyl alcohol (balance water) was used instead of 70% ethyl alcohol. The results are set forth in Table 3. In all instances, the Reb D content increased as a result of the trituration. In Examples 3.2 and 3.3, the Reb M content and the TSG content also increased. The recovery of Reb D and Reb M was substantially higher compared to Example 1.

TABLE 3

| | Crude | Triturated Product | | | Triturated Product | | |
|---|---|---|---|---|---|---|---|
| | Composition | Composition (wt. %) | | | Recovery (%) | | |
| | Composition | Ex. 3.1 | Ex. 3.2 | Ex. 3.3 | Ex. 3.1 | Ex. 3.2 | Ex. 3.3 |
| | (wt. %) | (120 g/L) | (170 g/L) | (250 g/L) | (120 g/L) | (170 g/L) | (250 g/L) |
| Reb D | 19.65 | 51.86 | 40.58 | 35.76 | 94.15 | 95.48 | 97.21 |
| Reb M | 15.10 | 7.8 | 23.28 | 24.25 | 18.12 | 71.83 | 87.12 |
| Reb A | 36.10 | 22.17 | 23.79 | 24.8 | 21.96 | 32.04 | 40.86 |

| | | | | | |
|---|---|---|---|---|---|
| TSG* | 89.78 | 89.59 | 95.83 | 93.31 | N/A |

*Composition of other steviol glycosides e.g. Stevioside, Rebaudioside F, Rebaudioside C, Dulcoside A, Rubusoside, Rebaudioside B are not reported in Table 3 but are included in TSG.

### EXAMPLE 4

### Trituration with 95% Ethyl Alcohol Solvent

Example 1 was repeated with the difference that 95% ethyl alcohol (balance water) was used instead of 70% ethyl alcohol. The results are set forth in Table 4. In all instances, the content of Reb D and TSG increased as a result of the trituration.

TABLE 4

| | Crude | Triturated Product | | | Triturated Product | | |
|---|---|---|---|---|---|---|---|
| | composition | Composition (wt. %) | | | Recovery (%) | | |
| | Composition | Ex. 4.1 | Ex. 4.2 | Ex. 4.3 | Ex. 4.1 | Ex. 4.2 | Ex. 4.3 |
| | (wt. %) | (120 g/L) | (170 g/L) | (250 g/L) | (120 g/L) | (170 g/L) | (250 g/L) |
| Reb D | 19.65 | 41.24 | 39.94 | 36.22 | 93.72 | 93.39 | 95.25 |
| Reb M | 15.10 | 11.21 | 11.67 | 12.31 | 31.66 | 36.06 | 42.92 |
| Reb A | 36.10 | 30.45 | 31.16 | 33.44 | 36.27 | 40.54 | 48.92 |

| | | | | | |
|---|---|---|---|---|---|
| TSG* | 89.78 | 93.42 | 93.92 | 94.73 | N/A |

*Composition of other steviol glycosides e.g. Stevioside, Rebaudioside F, Rebaudioside C, Dulcoside A, Rubusoside, Rebaudioside B are not reported in Table 4 but are included in TSG.

### EXAMPLE 5

### Two Triturations with 80% Ethanol

170 g/L of amorphous steviol glycoside powder (crude composition) was mixed with 80% ethanol (balance water) to produce a slurry. The slurry was stirred at 700 rpm for 1 hour at room temperature, filtered on a buchner funnel with a Whatman 5 filter, and washed with cold anhydrous 200 proof ethanol. The resulting solid was dried and then re-slurried at a concentration of 170 g/L in 80% ethanol (balance water). The second slurry was stirred at 700 rpm for 1 hour at room temperature, filtered on a buchner funnel with a Whatman 5 filter, and washed with cold anhydrous 200 proof ethanol. The resulting solid was dried and analyzed by HPLC using the 2017 JECFA monograph for steviol glycosides.

The results are set forth in Table 5. The content of Reb D, Reb M and TSG increased as a result of the first and second trituration.

TABLE 5

| | Crude | First Triturated | Second Triturated | |
|---|---|---|---|---|
| | composition | product | product | |
| | Composition | Composition | Composition | Recovery |
| | (wt. %) | (wt. %) | (wt. %) | (%) |
| Reb D | 19.65 | 46.24 | 55.03 | 84.50 |
| Reb M | 15.10 | 27.38 | 31.50 | 64.46 |
| Reb A | 36.10 | 17.11 | 9.08 | 7.99 |
| TSG | 89.78 | 96.01 | 97.22 | N/A |

* Composition of other steviol glycosides e.g. Stevioside, Rebaudioside F, Rebaudioside C, Dulcoside A, Rubusoside, Rebaudioside B are not reported in Table 6 but are included in TSG.

### EXAMPLE 6

### Crystallization of Liquid Filtrate

The liquid filtrate from Example 2.1 (120 g/L) was used as feed for crystallization. A mixture of 95% pure Rebaudioside D and Rebaudioside M powder were added to facilitate crystallization. This slurry was controlled at 1° C. for 72 hours. After 72 hours, the slurry was filtered using a buchner funnel and washed with 95% pure ethyl alcohol. The filter cake was oven dried at 50° C. for approximately 16 hours, and subsequently analyzed by HPLC using 2017 JECFA monograph for steviol glycosides. The results of this analysis are set forth in Table 6.

TABLE 6

| | Crystalline | | Overall (trituration |
|---|---|---|---|
| | Product | Recovery | and crystallization) |
| | Composition | crystallization | recovery |
| | (wt. %) | (%) | (%) |
| Reb D | 21.93 | 65.22 | 91.39 |
| Reb M | 61.77 | 68.95 | 73.27 |
| Reb A | 12.25 | 4.27 | 18.06 |

| | | |
|---|---|---|
| TSG* | 97.47 | N/A |

*Composition of other steviol glycosides e.g. Stevioside, Rebaudioside F, Rebaudioside C, Dulcoside A, Rubusoside, Rebaudioside B are not reported in Table 2A but are included in TSG.

### EXAMPLE 7

### Crystallization of Liquid Filtrate

Example 6 was repeated with the difference that the liquid filtrate from Example 2.2 (170 g/L) was used as feed for crystallization. The results are set forth in Table 7.

TABLE 7

| | | | Overall (trituration |
|---|---|---|---|
| | | Recovery | and crystallization) |
| | Composition | crystallization | recovery |
| | (wt. %) | (%) | (%) |
| Reb D | 20.49 | 62.38 | 91.06 |
| Reb M | 66.68 | 88.01 | 75.18 |
| Reb A | 7.43 | 3.39 | 17.96 |

| | | |
|---|---|---|
| TSG* | 95.40 | N/A |

*Composition of other steviol glycosides e.g. Stevioside, Rebaudioside F, Rebaudioside C, Dulcoside A, Rubusoside, Rebaudioside B are not reported in Table 2B but are included in TSG.

### EXAMPLE 8

### Crystallization of Liquid Filtrate

The liquid filtrate from Example 2.2 (170 g/L) was used as feed for crystallization. This slurry was controlled at 25° C. for 72 hours. After 72 hours, the slurry was filtered using buchner funnel and washed with 95% pure ethyl alcohol. The filter cake was oven dried at 50° C. for approximately 16 hours, and subsequently analyzed by HPLC using 2017 JECFA monograph for steviol glycosides. The results are set forth in Table 8.

TABLE 8

| | Crystalline | | Overall (trituration |
|---|---|---|---|
| | Product | Recovery | and crystallization) |
| | composition | crystallization | recovery |
| | (wt. %) | (%) | (%) |
| Reb D | 22.19 | 59.82 | 91.21 |
| Reb M | 65.71 | 66.37 | 73.03 |
| Reb A | 8.58 | 2.73 | 17.89 |

| | | |
|---|---|---|
| TSG* | 97.04 | N/A |

*Composition of other steviol glycosides e.g. Stevioside, Rebaudioside F, Rebaudioside C, Dulcoside A, Rubusoside, Rebaudioside B are not reported in Table 2C but are included in TSG.

1. A method for purifying a crude composition comprising one or more steviol glycosides, said method comprising: (a) providing a crude composition comprising one or more steviol glycosides, wherein said composition is in amorphous form; (b) subjecting said crude composition to a trituration step, said trituration step comprising contacting said crude composition with a polar solvent, resulting in a triturated product; and (c) recovering said triturated product.

2. The method according to claim 1, wherein said providing said crude composition comprises spray drying a solution comprising said one or more steviol glycosides in dissolved form.

3. (canceled)

4. The method according to claim 1, wherein said one or more steviol glycosides comprises Rebaudioside B, Rebaudioside D, Rebaudioside M, or a mixture thereof.

5. The method according to claim 1, wherein said one or more steviol glycosides comprises Rebaudioside B, wherein said crude composition comprises at least 5 wt. % or at least 10 wt. % of Rebaudioside B on a dry weight basis.

6. The method according to claim 1, wherein said one or more steviol glycosides comprises Rebaudioside D, wherein said crude composition comprises at least 5 wt. % or at least 10 wt. % of Rebaudioside D on a dry weight basis.

7. The method according to claim 1, wherein said one or more steviol glycosides comprises Rebaudioside M, wherein said crude composition comprises at least 5 wt. % or at least 10 wt. % of Rebaudioside M on a dry weight basis.

8. The method according to claim 1, wherein said one or more steviol glycosides comprises Rebaudioside A, wherein said crude composition comprises at least 10 wt. % or at least 20 wt. % of Rebaudioside A on a dry weight basis.

9. The method according to claim 1, wherein said crude composition has a total content of steviol glycosides of at least 60 wt. % on dry weight basis.

10. (canceled)

11. The method according to claim 1, wherein said solvent comprises water, an alcohol, or a mixture thereof.

12.-21. (canceled)

22. The method according to claim 1, wherein the content of Rebaudioside B in the triturated product on a dry weight basis is at least 5 wt. % (absolute), at least 10 wt. % (absolute), or at least 15 wt. % (absolute) higher than the content of Rebaudioside B in the crude composition on a dry weight basis.

23. The method according to claim 1, wherein the content of Rebaudioside D in the triturated product on a dry weight basis is at least 5 wt. % (absolute), at least 10 wt. % (absolute), or at least 15 wt. % (absolute) higher than the content of Rebaudioside D in the crude composition on a dry weight basis.

24. The method according to claim 1, wherein the content of Rebaudioside M in the triturated product on a dry weight basis is at least 5 wt. % (absolute), at least 10 wt. % (absolute), or at least 15 wt. % (absolute) higher than the content of Rebaudioside M in the crude composition on a dry weight basis.

25. The method according to claim 1, wherein the content of Rebaudioside A in the triturated product on a dry weight basis is at least 5 wt. % (absolute) lower than the content of Rebaudioside A in the crude composition on a dry weight basis, preferably at least 10 wt. % (absolute) lower, more preferably at least 15 wt. % (absolute) lower.

26. The method according to claim 1, wherein the total steviol glycoside content in the triturated product on a dry weight basis is at least 3 wt. % (absolute), at least 4 wt. % (absolute), or at least 5 wt. % (absolute) higher than the total steviol glycoside content in the crude composition on a dry weight basis.

27. The method according to claim 1, wherein the trituration step comprises obtaining a slurry comprising said crude composition and said solvent, and wherein said recovering of said triturated product comprises separating said triturated product from said slurry, optionally wherein said triturated product is separated from said slurry by filtration or centrifugation.

28. The method according to any preceding claim 1, wherein the method further comprises:
(d) recovering the solvent from the trituration step and subjecting said recovered solvent to one or more crystallization steps to obtain crystalline steviol glycosides comprising crystalline Rebaudioside B, crystalline Rebaudioside D, crystalline Rebaudioside M, crystalline Rebaudioside A, or mixtures thereof.

29. The method according to claim 1, wherein the method further comprises:
(e) recovering the mother liquor from the one or more crystallization steps of (d) and subjecting said recovered mother liquor to one or more crystallization steps to obtain crystalline steviol glycosides comprising Rebaudioside A.

30. (canceled)

31. (canceled)

32. A steviol glycoside composition in amorphous form comprising:
from 10 to 60 wt. % of Rebaudioside D on a dry weight basis;
from 5 to 30 wt. % of Rebaudioside M on a dry weight basis; and
from 1 to 60 wt. % of Rebaudioside A on a dry weight basis.

33. The steviol glycoside composition according to claim 32, wherein the total steviol glycoside content in the composition is at least 95 wt. % on a dry weight basis.

34. (canceled)

35. A method for preparing a food product, for instance a beverage, said method comprising incorporating the composition according to claim 32 in said food product.
